(19) United States
(12) Patent Application Publication
(10) Pub. No.: US 2022/0223284 A1
(43) Pub. Date: Jul. 14, 2022

(54) SYSTEMS AND METHODS FOR MODELLING A HUMAN SUBJECT

(71) Applicant: KONINKLIJKE PHILIPS N.V., Eindhoven (NL)

(72) Inventors: Murtaza Bulut, Eindhoven (NL); Lieke Gertruda Elisabeth Cox, Eindhoven (NL); Cornelis Petrus Hendriks, Eindhoven (NL); Valentina Lavezzo, Eindhoven (NL)

(21) Appl. No.: 17/146,535

(22) Filed: Jan. 12, 2021

(51) Int. Cl. G16H 50/20; G06F 30/20

(52) U.S. Cl. CPC G16H 50/20; G06F 30/20

(57) ABSTRACT

Systems and methods are proposed for assessing digital twin accuracy by processing first and second input data to generated target output data and supplementary output data, respectively. Based on the target output data and the supplementary output data, a value of accuracy of the DT may be determined.

### FIELD OF THE INVENTION

The invention relates to modelling human subjects, and more particularly to the field of models of biological function (commonly referred to as digital twins).

### BACKGROUND OF THE INVENTION

A recent development in healthcare is the so-called ‘digital twin’ concept. In this concept, a digital representation or computational simulation (i.e. the Digital Twin (DT)) of a physical system is provided and connected to its physical counterpart, for example through the Internet of things as explained in US 2017/286572 A1 for example. Through this connection, the DT typically receives data pertaining to the state of the physical system, such as sensor readings or the like, based on which the digital twin can predict the actual or future status of the physical system, e.g. through simulation.

Such DT technology is also becoming of interest in the medical field, as it provides an approach to more efficient medical care provision. For example, a DT may be built using imaging data of a subject (i.e. patient), e.g. a person suffering from a diagnosed medical condition as captured in the imaging data.

The DT(s) of a subject (i.e. subject-specific computational simulations) may serve a number of purposes. Firstly, the DT(s) (rather than the patient) may be subjected to a number of virtual tests, e.g. treatment plans, to determine which treatment plan is most likely to be successful to the patient. This reduces the number of tests that physically need to be performed on the actual patient. Secondly, the DT(s) of a subject may be used to predict the onset, treatment (outcome) or development of medical conditions of the subject. That is, the DT(s) of a subject may offer a healthcare professionals advanced visualization and/or physical insights into health information of the subject, thus supporting improved Clinical Decision Support (CDS).

Typically, there will be multiple DTs associated with a single subject. These include DTs of individual organs, DTs of biological processes, and potentially DTs of behavior and psychology. The DTs will be different in terms of their output trustworthiness, output bias, speed of operation, needed resources, needed input, and other parameters related to the DT building and usage. The DTs will also be different in terms of user preferences, how users use them, and other human (preference and behavior) related factors.

Thus, there is a need for a structured governance policy, in order to cope with different DT characteristics. In particular, an important limitation is DT bias (or unfairness) that may be caused by the data used for training, validating, and testing. It is therefore desirable to develop a governance policy that can manage the operation of DTs so the biased outputs are minimized, and identification of biases are easier.

### SUMMARY OF THE INVENTION

The invention is defined by the claims.

According to examples in accordance with an aspect of the invention, there is provided a method for assessing a value of accuracy of a first digital twin of a biological system of a subject, the method comprising:

processing first input data with the first digital twin to generate target output data;

processing second input data with the first digital twin to generate supplementary output data; and

determining a value of accuracy of the first digital twin based on the target output data and supplementary output data.

Embodiments propose concepts for assessing accuracy of a first DT of a biological system of a subject. Such proposals are based on a realization that systems have limitations. This is also the case for the DTs. It is proposed that an important limitation for the data-driven systems is bias (or unfairness), and that bias is mainly due to the data used (for training, validating, and testing the system). Vast amount of data, and the way the data is utilized (for example by training convolutional neural networks, which are very difficult to interpret) makes it practically impossible to identify the source of bias, exactly quantify, or get rid of the bias. Embodiment thus propose one or more concepts for assessing accuracy of a first digital twin. Based on such assessed accuracy, a (governance) policy relating to the management of DTs with different fairness may be realised. For example, minimization of DT output biases and/or early detection of biases may reduce clinical errors, thus helping to improve patient care. Embodiments may therefore help reduce requirements placed on medical care resources.

As a result, systems and methods are proposed for assessing DT accuracy by processing first and second input data to generated target output data and supplementary output data, respectively. Based on the target output data and the supplementary output data, a value of accuracy of the output of a DT may be determined. Further, embodiments may facilitate tracking or monitoring of DT accuracy where a subject's condition is continuously changing.

Proposed embodiments may thus provide dynamic accuracy assessment concepts that cater for changing parameters and/or conditions.

By way of example, the following two exemplary cases may be considered.

In the first case, the first input data and second input data are the same type (e.g. come from the same sensors, or source), but collected at different times. For instance, the first input may include data for a 1^{st} hour (i.e. Hour1), and second input may include data for a 2^{nd} hour (i.e. Hour2).

In the second case, the first input data and second input data may differ in terms of the data size, or amount. In other words, the first input data and second input data may include the same type of data, coming from the same source, but the second input data may be a subset or superset of the first input data. For instance, the first input data may be data for two hours of measurements, and the second input data may be for one hour of measurements (in which case, there is one hour overlap between the first and second input data).

That is, the first and second input data may be linked in terms of the time period during which are acquired and/or in terms of the amount of data.

It is also proposed that operations of the DT may be controlled based on an accuracy determined according to embodiment. Further, this may be extended to control operations of connected DTs (e.g. based on comparison of their assessed accuracies).

Embodiments may therefore be of particular use in relation to DTs that support clinical decision making. Exemplary usage applications may for example, relate to predicting the onset, treatment (outcome) or development of medical conditions and/or medical procedures. Embodiments may thus be of particular use in relation to medical care management and/or prediction.

In some embodiments, determining a value of accuracy of the first digital twin comprises: estimating a first value of accuracy of the first digital twin based on the target output; and modifying the first value of accuracy based on the supplementary output data to determine a value of accuracy of the first digital twin.

Some embodiments may further comprise modifying the second input data based on the determined value of accuracy of the first digital twin; processing the modified second input data with the first digital twin to generate modified supplementary output data; and determining an updated value of accuracy of the first digital twin based on the target output data and modified supplementary output data.

Proposed embodiments may further comprise: processing the first input data with a second digital twin relating to the biological system of the subject to generate second output data; and determining a value of accuracy of the first digital twin based on the target output data and second output data. Such embodiments may also comprise: comparing the supplementary output data and second output data to generate a comparison result, and determining a value of accuracy of the first digital twin may then be further based on the comparison result.

Furthermore, such embodiments may also comprise processing the target output data with the second digital twin relating to generate second target output data. Determining a value of accuracy of the first digital twin may then be further based on the second target output data.

Also, embodiments may further comprise processing the supplementary output data with the second digital twin to generate second supplementary output data. Determining a value of accuracy of the first digital twin may then be further based on the second supplementary output data.

Some embodiments may also comprise processing the target output data and the second output data with a third digital twin relating to the biological system of the subject to generate third output data. A value of accuracy of the first digital twin may then be determined further based on the third output data.

Proposed embodiments may also comprise: modifying the first input data based on the determined value of accuracy of the first digital twin; processing the modified first input data with the first and second digital twins to generate updated target output data and updated second target output data, respectively; and determining a value of accuracy of at least one of the first digital twin and second digital twin based on the updated target output data and updated second target output data.

According to examples in accordance with yet another aspect of the invention, there is provided a computer program product comprising computer program code means which, when executed on a computing device having a processing system, cause the processing system to perform all of the steps of the method described above.

According to another aspect of the invention, there is provided a system for assessing a value of accuracy of a first digital twin of a biological system of a subject. The system comprises: a processor arrangement configured to process first input data with the first digital twin to generate target output data, and to process second input data with the first digital twin to generate supplementary output data; and an analysis component configured to determine a value of accuracy of the first digital twin based on the target output data and supplementary output data.

In an embodiment, the system may be further configured to generate a control instruction for a sensor or medical equipment based on determining a value of accuracy of the first digital twin. In this way, a sensor and/or medical equipment may be controlled according to results generated by embodiments. Dynamic and/or automated control concepts may therefore be realized by proposed embodiments.

Further, proposed concepts may provide a clinical decision support comprising a system according to a proposed embodiment.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 depicts a Venn diagram illustrating an exemplary overlapping relationship between the outputs of three DTs;

FIG. 2 is a process flow illustration depicting the processing steps for validating the DT2(o2) of FIG. 1 according to a proposed concept;

FIG. 3 depicts a Venn diagram illustrating an exemplary overlapping relationship between the outputs of three DTs;

FIG. 4 is a flow diagram of a method for assessing a value of accuracy of a first digital twin of a biological system of a subject according to a proposed embodiment;

FIG. 5 is a simplified block diagram of a system according to an embodiment; and

FIG. 6 is a simplified block diagram of a computer within which one or more parts of an embodiment may be employed

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

The invention provides concepts for assessing DT accuracy. Such concepts may also be extended to comparing DTs and assessing fairness characteristics of connected DTs. Such concepts may employ processing first and second input data in a DT, wherein the first and second input data is linked (e.g. in terms of acquisition time, amount, acquisition location, subject activity, environmental parameters/conditions, operational parameters, source, etc.) For example, first and second input data may be linked by one or more sensors used for data collection, or by one or more algorithms used for data collection or synthesis. In another example, first and second input data may be linked through a part of the body from which the data has been collected. In yet another example, the first and second input data may be linked by a time/date the data was acquired/collected. Subject condition or activity may be another property that links the first and second input data.

By way of explanation of the proposed concept(s), three DTs will now be considered. In particular, the three DTs relate to the same subject/patient, although, for other embodiments, this may not necessarily be the case.

The first DT (DT1) is a DT of the subject's heart. The second DT (DT2) is a DT of the subject's lungs. The third DT (DT3) is a DT of the subject's arteries.

The three DTs differ with respect to (i) fairness (i.e., trust and accuracy) of their output, and (ii) speed of the output generation. One way to assess the fairness is based on the amount of representative training data, maturity, and history of the DT.

For instance, DT1 may have been trained using lots of data, and has a history of successful usages (e.g. generated output recommendations resulted in improvement). DT2 may have been trained using medium sized data, and has limited history. DT3 may have been trained on limited data, and has very limited history. For such a situation, the three DTs may differ with respect to (i) fairness (i.e., trust and accuracy) of their output, and (ii) speed of the output generation as shown in the following table, Table 1:

TABLE 1

| Criteria | DT ordering |
|---|---|
| Output Fairness (i.e. trust) | DT1 > DT2 > DT3 |
| Output Generation Speed | DT3 > DT2 > DT1 |

Table 1 above ranks the three DTs in terms of output fairness, and output generation speed. From the example of Table 1, it can be seen that the first DT (DT1) is the most trustworthy, and the third DT (DT3) is the fastest.

With all three DTs belonging to the cardiovascular system, it can be expected that some of the outputs that can be generated by the corresponding DTs are common to each other (i.e. the same).

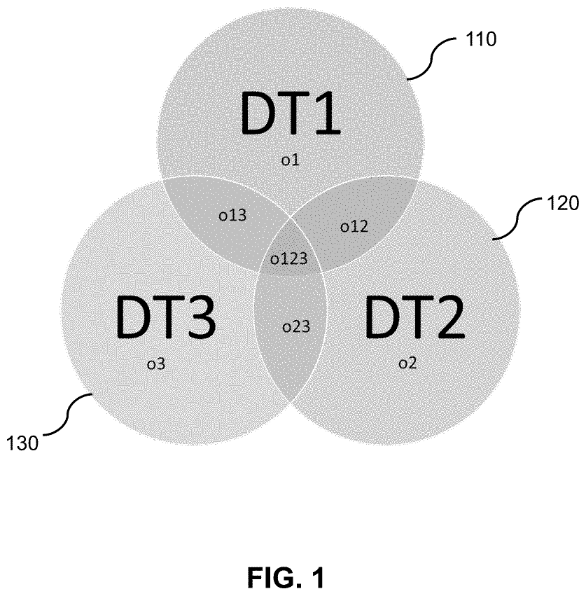

For example, referring now to FIG. 1, there is depicted a Venn diagram illustrating an exemplary overlapping relationship between the outputs of the three DTs.

The range of outputs for the first DT1, second DT2 and third DT3 DTs is illustrated by first 110, second 120 and third 130 circles, respectively. The overlap of circles illustrates where the DTs have common outputs.

For example, the region labelled “o12” relates to lung capacity and heart pumping volume. The region labelled “o13” relates to arteries taking blood away from heart (e.g. aspects of systemic circulation). The region labelled “o23” relates to arteries bringing blood to lungs (e.g. aspects of bronchial circulation). The region labelled “o123” relates to blood flow between lung and heart (e.g. aspects of pulmonary circulation).

Intersection between two DTs (e.g. DT1 and DT2) may be defined in the following three different ways:
Common outputs: This means that the two DTs can generate same outputs (which can be a signal, or data). The generated outputs can be compared;
Output of DT1 used as an input for DT2: In this case, an output generated by DT1 is used as one of the inputs for DT2, or can be used (by some predefined function) to generate some of the inputs for the DT2. For evaluation (of the validity of the DT1 output), the output of DT2 is evaluated (e.g. compared against a predicted or measured parameter);
Outputs of DT1 and DT2 are used as input to a third model, DT3: In the case, the DT1 and DT2 do not have any common outputs, or none of the outputs can be used as input to each other, their interaction can still be explored. This can be done via a third DT that uses outputs of DT1 and DT2 as two of its inputs, or uses them to derive some of its inputs. Similar to the second case, the validity of the DT1 and DT2 outputs, is assesses thorough the validity of the third digital twin, DT3, output.

Considering the exemplary bias ranking of Table 1 above, it is proposed to handle the outputs that can be generated by DT1 (i.e. the most trusted DT) as shown in the following table, Table 2:

TABLE 2

| Needed Output | DT1 output | DT2 output | DT3 output |
|---|---|---|---|
| o1 | o1 | None | None |
| o12 | o12 | None | None |
| o13 | o13 | None | None |
| o123 | o123 | None | None |

Table 2 illustrates an exemplary governance for outputs that can be generated by the most trusted DT (DT1).

In the first use case, shown in Table 2, if the required output can be generated by DT1, then there is no need to utilize other DTs (i.e. DT2 or DT3).

Conversely, for the outputs that cannot be generated by DT1, it is proposed to handle the outputs as shown in the following table, Table 3:

TABLE 3

| Needed Output | DT1 output | DT2 output | DT3 output | Rule |
|---|---|---|---|---|
| o2 | 012* | o2, 012* | none | R (o2) |
| o3 | 013* | none | o3, 013* | R (o3) |
| o23 | 0123* | 023, 0123* | none | R (o23) |

Table 3 illustrates an exemplary governance for outputs that cannot be generated by the most trusted DT (DT1). It is noted (using *) that more than one o12 or o13 outputs can be generated. The number of o12 or o13 outputs depends on the characteristics of the o2 and o3, respectively.

Further, the rules referred to in Table 3 above are detailed in the following table, Table 4:

TABLE 4

| Rule | Details |
|---|---|
| R(o2) | If f(DT1(o12), DT2(o12)) <= thr(o2) use o2, else |
| | regenerate* o2 and o12 until the condition of DT1 and DT2 |
| | output similarity is satisfied**. |
| R(o3) | If f(DT1(o13), DT2(o13)) <= thr(o3) use o3, else |
| | regenerate* o3 and o13 until the condition of DT1 and DT3 |
| | output similarity is satisfied**. |
| R(o23) | If f(DT1(o123), DT2(o123)) <= thr(o23) use o23, else |
| | regenerate* o23 and o123 until the condition of DT1 and |
| | DT2 output similarity is satisfied**. |

Table 4 above details exemplary rules for checking validity of outputs generated by the DTs (i.e. DT2 and DT3) other than the most trusted DT (i.e. DT1).

Here the aim is to quantitative compare two outputs that can be numerical or non-numerical values. Similarity (or difference) calculations are well-established, so detailed discussion of such is omitted.

However, purely by way of example, a technique for the comparison of numerical values (such as time domain signals, time series, images, or higher-dimensional data) may employ calculating similarity measures, such as cosine distance, Euclidian distance, correlation, intersection measures, etc. Also, the probability distribution of output1 (DT1(o12)) and output2 (DT2(o12)) may be compared to calculate a distance/similarity metric. Exemplary methods that may be employed may employ Kullback-Leibler divergence, Hellinger distance, various entropy metrics, and distance metrics. Such metrics can be extended to multi-variate probability distributions (where the outputs comprise multiple signals). Where case outputs are non-numerical (e.g. text), natural language based techniques can be employed, methods for measuring semantic textual similarity are well-developed field. Exemplary methods include entropy-based methods, topological and knowledge-based methods, latent semantic analysis, Jaro distance, Damerau-Levenshtein distance, block distance, cosine similarity, etc.

The result of the comparison function is a numerical value, typically between zero (0) and one (1), where zero indicates an exact match and one indicates no similarity (or vice versa, one=exact match, zero=no similarity, depending on the method employed).

The threshold value, which is determined as a function of the target output characteristics, is compared against the calculated distance (or similarity/difference) parameter. By way of example, proposed embodiments use the confidence level of the output to determine the threshold value. This can be done by defining a function that results in higher similarity requirements for lower confidence outputs. Assuming that a comparison function output equal to zero corresponds to exact match, an example function is as follows:

if output confidence level<=0.8, thr=−1 forcing regeneration of the output

if 0.8<output confidence level<=0.85, then thr=0 (require exact match),

else thr=1-output confidence level.

It is noted (using *) that, before regeneration, something has to be changed (such as using new data, a new model, or new settings), otherwise same outputs can be expected. It is also noted (using **) that, similarly to Table 3, multiple o12, o13, and o123 can be generated. In that case, R(o2), R(o3), and R(o23) need to apply to all of these, respectively.

The exemplary rules of Table 4 above indicate that intersecting outputs from two different DTs are compared (using one or more functions) with respect to a criterion (e.g. threshold) that is determined as a function of the required output. In other words, the required output characteristics determine how the intersecting outputs should be compared, and what an acceptable similarity or difference should be.

By way of example, one may consider two cases, where in the first case DT2(o2) is calculated with a first level of confidence (confidence1), and in the second case a different DT2(o2) is calculated with a second level of confidence (confidence2), where the first level of confidence is greater than the second level of confidence (i.e. confidence1>confidence 2). Due to the lower level confidence in the second case, the threshold function can force DT1(o12) and DT2(o12) to be more similar to each other (by utilizing stricter comparison criteria than first case), before accepting DT2(o2).

It addition to using the confidence level, other options may also be used. For example, for certain types of output such as numerical outputs, the threshold can be defined as a function of different features calculated from the output. For example, features such as frequency content, zero crossing rate, variation, mean, noise, type and amount of missing data can be calculated from the output and used for determination of the threshold. Additionally, a rule-based method where output features will be compared against specifications set by clinicians can be also considered. In one case, these rules can be implemented as a look-up table, or decision tree.

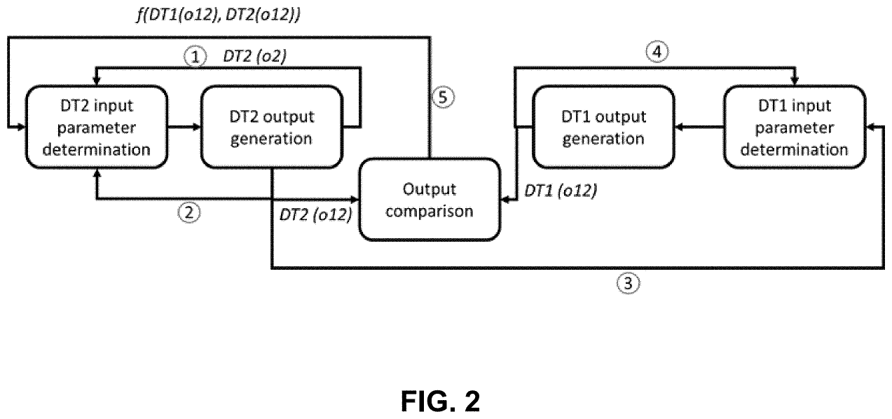

Referring now to FIG. 2, there are depicted the processing steps for validating the DT2(o2) according to a proposed concept, based on the similarity function ƒ for evaluating the difference between DT1(o12) and DT2(o12).

In the first step (labelled with a circle containing “1”), the second DT (DT2) input parameters that enable generation of o2 from the second DT (i.e. DT2(o2)) are determined and set. The output DT2(o2) is generated, and, if necessary, the input parameter can be re-tuned based on the generated output. Thus, a standard feedback loop may be implemented to tune the input parameter(s) for the second DT.

In the second step (labelled with a circle containing “2”), new or additional input parameters for the second DT (DT2) that enable generation of DT2(o12) from DT2 are determined and set. Again, re-tuning of the input parameters can be undertaken based on DT2(o12) and DT2(o2). In other words, the inputs to the second DT (DT2) can be retuned so that both DT2(o2) and DT2(o12) satisfy a predetermined quality criterion.

It is noted that, in an alternative implementation, the process of input parameter setting and re-tuning for DT2(o2) and DT(o12) can be done in a single step, e.g. if they have one or more input parameters in common.

In the third step (labelled with a circle containing “3”), the input parameters for the first DT (DT1) that are required to generate DT1(o12) are determined based on the DT2(o12) output characteristics. For example, DT1(o12) should contain the same type of signals, or data (e.g. statistics) to enable comparison to DT2(o12).

In the fourth step (labelled with a circle containing “4”), the input parameters for the first DT (DT1) that are required to generate DT1(o12) are re-tuned based on the DT1(o12) output characteristics. This again resembles a standard feedback loop, and its purpose is to optimize the DT1(o12).

It is noted that, in an alternative implementation, the determination and tuning of DT1 parameters may be undertaken by simultaneous processing of DT1(o12) and DT2(o12) output requirements.

In the fifth step (labelled with a circle containing “5”), the comparable outputs (i.e. outputs of the same nature/type which can be quantitatively compared) generated by the first DT1 and second DT2 DTs are compared using a comparison function ƒ The resulting difference information is used as an input to DT2, where the information is used to re-tune the input parameters (if needed), until the DT2(o2) driven criterion function is satisfied.

By way of further explanation, we will now consider an example illustrating the connection between different digital models. Specifically, pulmonary circulation connects heart (DT1) and lungs (DT2); deoxygenated blood leaves the heart through the right ventricle through the pulmonary artery, and, in the lungs, the blood is oxygenated returned to the left atrium and ventricle of the heart.

Further, pleural pressure is transmitted to both organs, resulting in comparable pressure and volume changes in the lungs, and chambers of the heart. An important component that has direct influence on this interaction is the ventricular interaction, resulting from the flexible septum (the stout wall separating the ventricles) and elastic passive pericardium (double-walled sac containing the heart and the roots of the great vessels).

Using, anatomic structure properties, such as the parameters relating to the septum for example, it is possible to define intersections (i.e. interactions) between the digital twin models of heart (e.g. DT1) and lungs (e.g. DT2). Due to ventilation (respiration), the left stroke volume, and right ventricle stroke volume (SV) are disassociated (i.e. respond differently). For example, during (early) expiration, the left ventricle SV increases, whereas the right ventricle SV decreases. These variations become bigger with the increase in the amplitude of the pleural pressure variations.

Heart activity introduces volume and pressure variations in the rib cage, which results in varying lung (thoracic) volume. Based on this, rib cage variations can be used to connect both models (e.g. DT1 and DT2) (in addition to the interactions described in the previous paragraph).

To combine the heart and lung models (DT1 and DT2), the alveolar, pleural, and abdominal pressure parameters can be used. The heart DT1 and lungs DT2 models can both generate estimates of these, and the outputs can be compared. Alternatively, the lungs model DT2 can generate these parameters, and they can be used as input to the heart model DT1. The resulting output of the heart model DT1 can be compared to the other outputs of the DT1.

Intrathoracic blood volume is another parameter that can be used. Differently from the previous parameters described above, this parameter can be calculated by the heart DT (i.e. DT1) and used in the simulations of the lung DT (i.e. DT2). As indicated above, alternatively, both the heart DT1 and lungs DT2 models can calculate this parameter, which then can be compared.

Accordingly, by way of summary, different parameters concerning two DTs can be used to connect both DTs. These parameters can be established based on the anatomical connections, for example. That is, in case of the anatomically inspired connections, prior earnings and observations are used. For instance, since both the heart and lungs reside in the rib cage, it can be expected that they are connected due to this physical property. Or as in the case of septum, their connections can be based on the observation that septum properties can link two models (because blood from heart to lungs comes from the right ventricle, and blood from lungs to heart comes from the left atrium and ventricle, meaning that septum properties can influence blood flow dynamics between the two systems). In summary, aspects related to the physical location of both organs, or the blood flow dynamics between them can be used to infer or determine connections between DTs.

Alternatively, interactions may be identified via analysis of data (including signals, statistics, user-entered notes, etc.) generated by both DTs. For example, connections (i.e. interactions) can be determined based on analysis of the data generated by two DTs. Analysis, such as statistical techniques based on correlation, causation, regression, etc., or machine learning driven techniques may be employed. In the former case, it may be observed that outputs of first DT1 and second DT2 digital twin models correlate with each other, and the correlation (or regression) metric can be used to perform checks. In the latter case, outputs of the first DT1 and second DT2 digital twin models can be used an input to a machine learning algorithm, and the output of the machine learning algorithm can be assessed for validating DT1 and DT2 outputs.

By way of further explanation, we will now consider: DTs with non-overlapping outputs. Although all human organs are interconnected (and thus in practice it is hard to imagine that there will not be common outputs (or interactions) between two human-organ related DTs), there may be specific cases where the DT outputs are limited (or DTs are used for modelling specific functionalities wherein outputs are non-overlapping (i.e., DT1 and DT4 are mutually exclusive). In that case, one option is to use known and validated mathematical models of the human body and its functions to compare the outputs of DT1 and DT4.

Using the relevant mathematical model, and using the output generated by the less trusted DT (i.e. DT4) as one of the model input parameters, an estimate of the trusted DT output (i.e. DT1) output is generated. The estimate is compared to the output generated by the DT1, and if they are sufficiently similar then output of DT4 is accepted, otherwise regenerated.

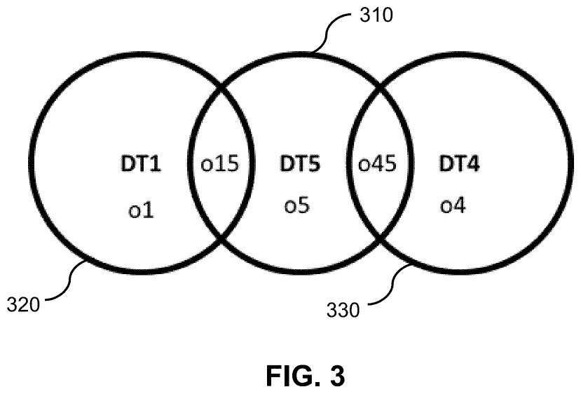

Another approach is to construct a new DT (e.g. DT5) that has a range of outputs 310 intersecting with the range 320 outputs of DT1 and the range 330 of outputs of DT4 as shown in FIG. 3. Once this is done, the policy described in the previous section can be applied. Assuming that DT1 is more trusted that DT4. For instance:
(i) If the required output is o1, generate DT1(o1).
(ii) If the required output is o4, generate DT4(o4), DT4(o45), DT5(o45),DT5(o5), DT5(o15), and DT1(o15), in the stated order. Accept DT4(o4) if R(o4) is satisfied, wherein R(o4) is as follows: (f(DT4(o45), DT5 (o45)) x g(DT1(o15), DT5(o15)))<thr(o4,o5), wherein g is a function, similar to f, comparing the two outputs.

DT4(o4) determines what o45 should contain. o45 determines what o5 should contain, and o5 determines what o15 should contain.

As a feedback step: o45 (from both DT4 and DT5), o5 and o15 (from both DT5 and DT1) can be optimized by feeding the result back to the module that determines o4.

Once a DT is constructed it can be used to test or predict different conditions/parameters related to a physical object (i.e. organ). For example using DT of heart, many parameters/conditions can be tested (e.g. condition of right/left ventricles, muscle strength, timing, etc.). This is what differentiates DTs from typical data-based only systems, where only a single or few selected conditions are modelled (i.e. output is related only to what the model has been trained for). The advantage of DTs is that they can be built so that there is a large overlap with other DTs (in terms of input needs, and generated output), to allow for the solution we describe. Intersection with other DTs can be an important additional design parameter, or a feature of a DT (similar to redundancy (intentionally introduced) in the communication protocols). Indeed, considering that the expected future developments of AI-based systems, ensuring overlapping inputs and outputs may be used to ensure automated and continuous accuracy verifications and checks across different systems.

By way of further explanation of the proposed concept(s), several additional embodiments will now be described, which can be taken as extension or alternatives to what has been described above.

Firstly, an alternative utilization of DTs for the required outputs in Table 2 above is considered. Differently from what is shown in Table 2 above, if the time and resources allow, all related DTs can be used to generate outputs as shown in the following table, Table 5:

TABLE 5

| Required Output | DT1 output | DT2 output | DT3 output |
|---|---|---|---|
| o1 | o1 | None | None |
| o12 | o12 | o12 | None |
| o13 | o13 | None | o13 |
| o123 | o123 | o123 | o123 |

Table 5 above depicts an example wherein all related DTs are configured to generate output regardless of the fairness.

The advantage of this approach is that it allows for the fairness levels of the less-trusted DTs (e.g. DT2 and DT3) to be updated based on the comparison of the outputs with the DT1 output. For example, if the DT2 output is consistently similar to DT1 output, the DT2 fairness level indicators may be set to higher values.

Moreover, having more outputs generated (and hence more information) can also prompt a user to assess DT1 outputs differently and critically, which may help in identifying DT1 output biases, and hence further improve DT1.

Another option to help improve DT2 and DT3 fairness is forcing DT2 and DT3 to generate outputs at all times where a DT1 output can be linked to any of the DT2 or DT3 outputs. For example, if the required output is o1, DT1(o1) will be generated. In this embodiment, it is also proposed to generate the following outputs: DT1(o12), DT2(o12), DT1(o13), DT3(o13),DT1(o123), DT2(o123), DT3(o123) (together with the DT(o1)). This allows for more frequent and detailed inspections of the less trusted DT2 and DT3, in relation to the more trusted DT1. This approach may result in faster and better identification, along with improvement in DT2 and DT3 fairness.

It is also noted that trust and speed relations between DTs may not be static, and they may be also output requirement (i.e. DT usage) dependent. For example, for certain output types, DT3 may be more trusted than DT1. For instance, hemodynamic models (e.g. DT3) outputs may be better representative of vagus nerve functions than the heart modelling, or abdominal pressure can be better modelled based on the output of lungs model (e.g. DT2) than output of heart model (e.g. DT1). The determination of the trust relation in this case can be based on a look up table, where for different outputs (i.e. DT usages), different trust relationships (e.g. rankings) can be available.

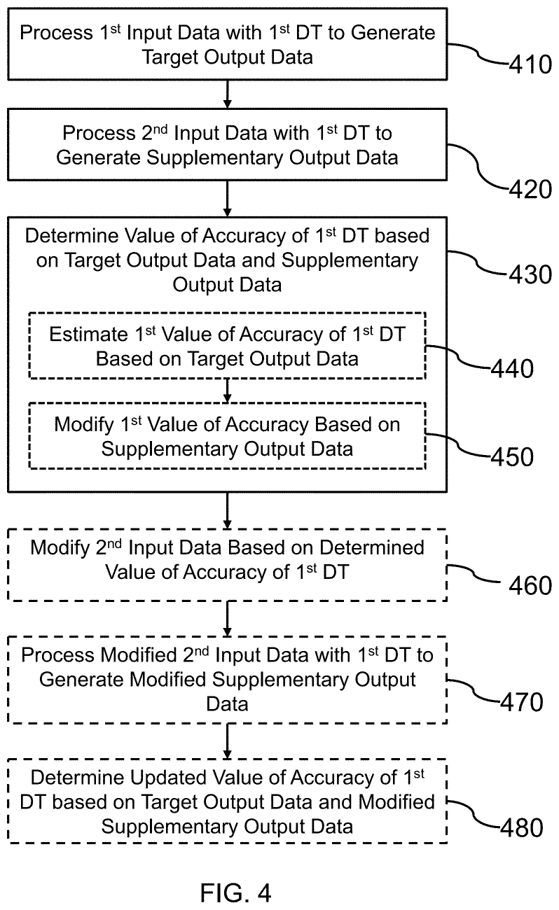

Referring now to FIG. 4, there is depicted a flow diagram of a method for assessing a value of accuracy of a first digital twin of a biological system of a subject according to a proposed embodiment.

Step 410 comprises processing first input data with the first DT to generate target output data.

Step 420 comprises processing second input data with the first DT to generate supplementary output data.

The first input data and second input data are linked by at least one known factor. That is, the first and second input data have one or more parameters, characteristics or attributes in common, such as time, location, source, subject, size, data field(s), data amount, etc. In this exemplary method, the first input data and second input data are of the same type (e.g. come from the same sensors, or source), but relate to different times (i.e. have been acquired over two differing time period). For instance, the first input data may include data for a 1^{st} time window of data acquisition, and the second input data may include data for a 2^{nd}, different time window of data acquisition.

Step 430 comprises determining a value of accuracy of the first DT based on the target output data and supplementary output data. Specifically, in this exemplary method, step 430 of determining a value of accuracy of the first DT comprises sub-steps 440 and 450. Step 440 comprises estimating a first value of accuracy of the first DT based on the target output. Then, in step 450, the first value of accuracy is modified based on the supplementary output data, so as to determine a value of accuracy of the first DT.

The exemplary embodiment of FIG. 4 may also include further steps to subsequently refine/update the value of accuracy (determined from completion of steps 410, 420 and 430. The further steps are indicated by dashed boxes in FIG. 4 and comprise steps 460, 470, and 480.

Step 460 comprises modifying the second input data based on the determined value of accuracy of the first DT. Then, in step 470, the modified second input data is processed with the first DT to generate modified supplementary output data. Finally, in step 480, an updated value of accuracy of the first DT is determined based on the target output data and modified supplementary output data.

It is to be understood that the method of FIG. 4 is only illustrative and the ordering of one or more of the steps may be modified in alternative embodiments. For example, steps 410 and 420 may be undertaken in parallel.

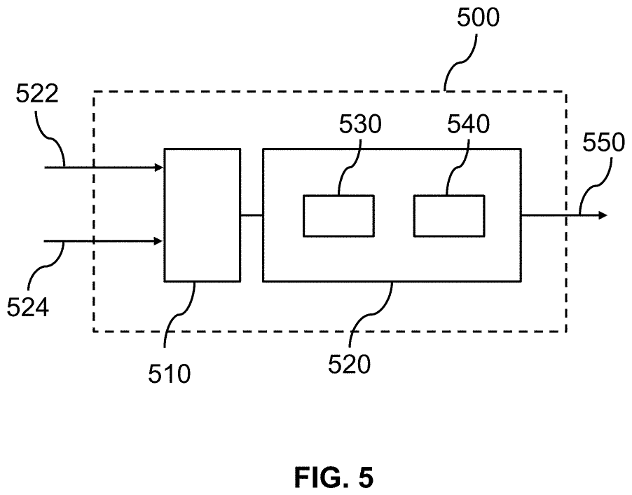

Referring to FIG. 5, there is shown a simplified block diagram of a system 500 according to an embodiment. The system 500 is for assessing a value of accuracy of a first DT of a biological system of a subject and comprises a processor 510 and an analysis component 520.

The processor 510 is configured to receive first input data 522 and second input data 524 (e.g. via an input interface).

The first input data 522 and second input data 524 have one or more parameters, characteristics or attributes in common. In this exemplary method, the first input data 522 and second input data 524 include the same type of data, coming from the same source, but the second input data 524 is a superset of the first input data 522. That is, second input data 524 consists of the first input data 522 plus further data from the same data source. For instance, the first input data 522 may be data for two hours of measurements from a sensor, and the second input data 524 may comprise data for four hours of measurements from the sensor (wherein the four hours include the two hours.

The processor 510 is configured to process the first input data 522 with the first DT to generate target output data, and to process the second input data 524 with the first DT to generate supplementary output data.

The analysis component 520 receives the target output data and the supplementary output data from the processor 510. Based on the target output data and supplementary output data, the analysis component determines a value of accuracy of the first DT. Specifically, the analysis component 520 comprises an estimation unit 530 and a modification component 540. The estimation unit 530 is configured to estimate a first value of accuracy of the first DT based on the target output. The modification component 540 then modifies the first value of accuracy based on the supplementary output data, so as to determine a value of accuracy of the first DT.

The analysis component 540 provides the determines value of accuracy of the first DT as an output signal 550 (e.g. to an display interface or device controller).

From the above description, it will be understood that the proposed concept(s) have a wide range of application possibilities related to the fairness evaluation and monitoring of automated (machine learning, or artificial intelligence) systems and algorithms. In particular, embodiments may be particularly suited to fields where a wrong output can have serious consequences (e.g. health care) and/or where fairness based evaluation is important.

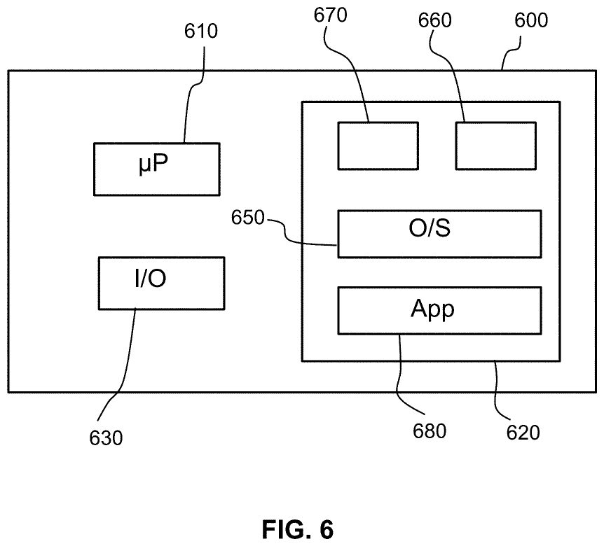

By way of further example, FIG. 6 illustrates an example of a computer 600 within which one or more parts of an embodiment may be employed. Various operations discussed above may utilize the capabilities of the computer 600. For example, one or more parts of a system for assessing a value of accuracy of a first digital twin of a biological system of a subject may be incorporated in any element, module, application, and/or component discussed herein. In this regard, it is to be understood that system functional blocks can run on a single computer or may be distributed over several computers and locations connected across a cloud-based system (e.g. connected via internet). That is, at least part of the DTs and data may be stored and executed in one or more cloud-based systems, of which the computer 600 may be part.

The computer 600 includes, but is not limited to, PCs, workstations, laptops, PDAs, palm devices, servers, storages, and the like. Generally, in terms of hardware architecture, the computer 600 may include one or more processors 610, memory 620, and one or more I/O devices 630 that are communicatively coupled via a local interface (not shown). The local interface can be, for example but not limited to, one or more buses or other wired or wireless connections, as is known in the art. The local interface may have additional elements, such as controllers, buffers (caches), drivers, repeaters, and receivers, to enable communications. Further, the local interface may include address, control, and/or data connections to enable appropriate communications among the aforementioned components.

The processor 610 is a hardware device for executing software that can be stored in the memory 620. The processor 610 can be virtually any custom made or commercially available processor, a central processing unit (CPU), a digital signal processor (DSP), or an auxiliary processor among several processors associated with the computer 600, and the processor 610 may be a semiconductor based microprocessor (in the form of a microchip) or a microprocessor.

The memory 620 can include any one or combination of volatile memory elements (e.g., random access memory (RAM), such as dynamic random access memory (DRAM), static random access memory (SRAM), etc.) and non-volatile memory elements (e.g., ROM, erasable programmable read only memory (EPROM), electronically erasable programmable read only memory (EEPROM), programmable read only memory (PROM), tape, compact disc read only memory (CD-ROM), disk, diskette, cartridge, cassette or the like, etc.). Moreover, the memory 620 may incorporate electronic, magnetic, optical, and/or other types of storage media. Note that the memory 620 can have a distributed architecture, where various components are situated remote from one another, but can be accessed by the processor 610.

The software in the memory 620 may include one or more separate programs, each of which comprises an ordered listing of executable instructions for implementing logical functions. The software in the memory 620 includes a suitable operating system (O/S) 650, compiler 660, source code 670, and one or more applications 680 in accordance with exemplary embodiments. As illustrated, the application 680 comprises numerous functional components for implementing the features and operations of the exemplary embodiments. The application 680 of the computer 600 may represent various applications, computational units, logic, functional units, processes, operations, virtual entities, and/or modules in accordance with exemplary embodiments, but the application 680 is not meant to be a limitation.

The operating system 650 controls the execution of other computer programs, and provides scheduling, input-output control, file and data management, memory management, and communication control and related services. It is contemplated by the inventors that the application 680 for implementing exemplary embodiments may be applicable on all commercially available operating systems.

Application 680 may be a source program, executable program (object code), script, or any other entity comprising a set of instructions to be performed. When a source program, then the program is usually translated via a compiler (such as the compiler 660), assembler, interpreter, or the like, which may or may not be included within the memory 620, so as to operate properly in connection with the O/S 650. Furthermore, the application 680 can be written as an object oriented programming language, which has classes of data and methods, or a procedure programming language, which has routines, subroutines, and/or functions, for example but not limited to, C, C++, C#, Pascal, BASIC, API calls, HTML, XHTML, XML, ASP scripts, JavaScript, FORTRAN, COBOL, Perl, Java, ADA, .NET, and the like.

The I/O devices 630 may include input devices such as, for example but not limited to, a mouse, keyboard, scanner, microphone, camera, etc. Furthermore, the I/O devices 630 may also include output devices, for example but not limited to a printer, display, etc. Finally, the I/O devices 630 may further include devices that communicate both inputs and outputs, for instance but not limited to, a NIC or modulator/demodulator (for accessing remote devices, other files, devices, systems, or a network), a radio frequency (RF) or other transceiver, a telephonic interface, a bridge, a router, etc. The I/O devices 630 also include components for communicating over various networks, such as the Internet or intranet.

If the computer 600 is a PC, workstation, intelligent device or the like, the software in the memory 620 may further include a basic input output system (BIOS) (omitted for simplicity). The BIOS is a set of essential software routines that initialize and test hardware at startup, start the O/S 650, and support the transfer of data among the hardware devices. The BIOS is stored in some type of read-only-memory, such as ROM, PROM, EPROM, EEPROM or the like, so that the BIOS can be executed when the computer 600 is activated.

When the computer 600 is in operation, the processor 610 is configured to execute software stored within the memory 620, to communicate data to and from the memory 620, and to generally control operations of the computer 300 pursuant to the software. The application 680 and the O/S 650 are read, in whole or in part, by the processor 310, perhaps buffered within the processor 610, and then executed.

When the application 680 is implemented in software it should be noted that the application 680 can be stored on virtually any computer readable medium for use by or in connection with any computer related system or method. In the context of this document, a computer readable medium may be an electronic, magnetic, optical, or other physical device or means that can contain or store a computer program for use by or in connection with a computer related system or method.

The application 680 can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

A single processor or other unit may fulfill the functions of several items recited in the claims.

It will be understood that the disclosed methods are computer-implemented methods. As such, there is also proposed a concept of a computer program comprising code means for implementing any described method when said program is run on a processing system.

The skilled person would be readily capable of developing a processor for carrying out any herein described method. Thus, each step of a flow chart may represent a different action performed by a processor, and may be performed by a respective module of the processing processor.

As discussed above, the system makes use of a processor to perform the data processing. The processor can be implemented in numerous ways, with software and/or hardware, to perform the various functions required. The processor typically employs one or more microprocessors that may be programmed using software (e.g. microcode) to perform the required functions. The processor may be implemented as a combination of dedicated hardware to perform some functions and one or more programmed microprocessors and associated circuitry to perform other functions.

Examples of circuitry that may be employed in various embodiments of the present disclosure include, but are not limited to, conventional microprocessors, application specific integrated circuits (ASICs), and field-programmable gate arrays (FPGAs).

In various implementations, the processor may be associated with one or more storage media such as volatile and non-volatile computer memory such as RAM, PROM, EPROM, and EEPROM. The storage media may be encoded with one or more programs that, when executed on one or more processors and/or controllers, perform the required functions. Various storage media may be fixed within a processor or controller or may be transportable, such that the one or more programs stored thereon can be loaded into a processor.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. If the term “adapted to” is used in the claims or description, it is noted that the term “adapted to” is intended to be equivalent to the term “configured to”. Any reference signs in the claims should not be construed as limiting the scope.

1. A method for assessing a value of accuracy of a first digital twin of a biological system of a subject, the method comprising:
processing first input data with the first digital twin to generate target output data;
processing second input data with the first digital twin to generate supplementary output data; and
determining a value of accuracy of the first digital twin based on the target output data and supplementary output data.

2. The method of claim 1, wherein determining a value of accuracy of the first digital twin comprises:
estimating a first value of accuracy of the first digital twin based on the target output; and
modifying the first value of accuracy based on the supplementary output data to determine a value of accuracy of the first digital twin.

3. The method of claim 1, further comprising:
modifying the second input data based on the determined value of accuracy of the first digital twin;
processing the modified second input data with the first digital twin to generate modified supplementary output data; and
determining an updated value of accuracy of the first digital twin based on the target output data and modified supplementary output data.

4. The method of claim 1, further comprising:
processing the first input data with a second digital twin relating to the biological system of the subject to generate second output data; and
determining a value of accuracy of the first digital twin based on the target output data and second output data.

5. The method of claim 4, further comprising:
comparing the supplementary output data and second output data to generate a comparison result, and
wherein determining a value of accuracy of the first digital twin is further based on the comparison result.

6. The method of claim 4, further comprising:
processing the target output data with the second digital twin relating to generate second target output data, and
wherein determining a value of accuracy of the first digital twin is further based on the second target output data.

7. The method of claim 4, further comprising:
processing the supplementary output data with the second digital twin to generate second supplementary output data, and
wherein determining a value of accuracy of the first digital twin is further based on the second supplementary output data.

8. The method of claim 4, further comprising:
processing the target output data and the second output data with a third digital twin relating to the biological system of the subject to generate third output data,
wherein determining a value of accuracy of the first digital twin is further based on the third output data.

9. The method of claim 4, further comprising:
modifying the first input data based on the determined value of accuracy of the first digital twin;
processing the modified first input data with the first and second digital twins to generate updated target output data and updated second target output data, respectively; and
determining a value of accuracy of at least one of the first digital twin and second digital twin based on the updated target output data and updated second target output data.

10. A computer program product comprising computer program code means which, when executed on a computing device having a processing system, cause the processing system to perform all of the steps of the method according to claim 1.

11. A system for assessing a value of accuracy of a first digital twin of a biological system of a subject, the system comprising:
a processor arrangement configured to process first input data with the first digital twin to generate target output data, and to process second input data with the first digital twin to generate supplementary output data; and
an analysis component configured to determine a value of accuracy of the first digital twin based on the target output data and supplementary output data.

12. The system of claim 11, wherein analysis component comprises:
an estimation unit configured to estimate a first value of accuracy of the first digital twin based on the target output; and
a modification component configured to modify the first value of accuracy based on the supplementary output data to determine a value of accuracy of the first digital twin.

13. The system of claim 11, wherein the processor arrangement is further configured to:
process the first input data with a second digital twin relating to the biological system of the subject to generate second output data; and
determine a value of accuracy of the first digital twin based on the target output data and second output data.

14. The system of claim 13, further comprising:
a comparison unit configured to compare the supplementary output data and second output data to generate a comparison result, and
wherein the analysis component is configured to determine a value of accuracy of the first digital twin further based on the comparison result.

15. A clinical decision support comprising a system for assessing a value of accuracy of a first digital twin of a biological system of a subject according to claim 11.
